(19) United States
(12) Patent Application Publication
(10) Pub. No.: US 2011/0311992 A1
(43) Pub. Date: Dec. 22, 2011

(54) Method of Prognosis and/or Diagnosing an IRS in a Patient Infected with M. Tuberculosis and Optionally HIV

(75) Inventor: Brigitte Autran, Paris (FR)

(73) Assignee: Institut National De La Sante Et De La Recherche Medicale (INSERM), Paris (FR)

(21) Appl. No.: 13/148,162

(22) PCT Filed: Feb. 5, 2010
(86) PCT No.: PCT/EP2010/051435

§ 371 Date: Sep. 7, 2011

(30) Foreign Application Priority Data
Feb. 6, 2009 (EP) 09305111.8

(51) Int. Cl. G01N 33/566

(52) U.S. Cl. 435/7.24

(57) ABSTRACT

The invention relates to a method of prognosing and/or diagnosing a M. tuberculosis-Immune restoration syndrome (TB-IRS) in an individual infected with M. tuberculosis and optionally HIV comprising comparing the percentage or concentration of γδ⁺ or γδ⁺Vδ2⁺T-lymphocytes which express an inhibitory KIR receptor with a predetermined value.

The invention relates to a method of prognosing and/or diagnosing a M. tuberculosis-Immune restoration syndrome (TB-IRS) in an individual comprising evaluating the percentage or concentration of γδ⁺ or γδ⁺Vδ2⁺T-lymphocytes which express an inhibitory KIR receptor with a predetermined value. In particular, the invention relates to a method of prognosing and/or diagnosing a TB-IRS in an individual co-infected with M. tuberculosis and HIV.

### BACKGROUND OF THE INVENTION

TB-IRS is observed in 5 to 10% of patients infected with Mycobacterium tuberculosis (TB) and can be induced by the administration of an anti-TB treatment. TB-IRS is more frequently observed in patients co-infected with HIV and M. tuberculosis (TB) after onset of Highly Active Antiretroviral Therapy (HAART) with a frequency up to 10 to 30% (Lawn et al. (2005) Lancet Infect Dis 5:361-373). TB-IRS is characterized by clinical symptoms revealing exacerbation of granulomatous lesions (Race et al. (1998) Lancet 351:252-255). The pathophysiology of TB-IRS is poorly described but is thought to reflect the sudden restoration of immune competence against M. tuberculosis. The TB-IRS diagnosis is currently solely based upon clinical findings and on microbiological exclusion criteria eliminating lack of control or relapses of TB.

At time of TB-IRS, recovery of strong Tuberculin-Skin-Test (TST) in previously anergic patients suggests the restoration of effective anti-mycobacterial immunity (Narita et al. (1998) Am J Respir Crit Care Med 158:157-161). Indeed, a strong increase of tuberculin-specific T-lymphocytes in patients developing TB-IRS has been demonstrated whereas non-TB-IRS patients did not (Bourgarit et al. (2006) Aids 20:F1-7). This phenomenon was associated with a massive inflammatory and Th1 cytokine storm. However, so far the nature of these IFN-γ producing cells is still unknown as is the hypothetical involvement of other cells playing a role in the IFN-γ and Th1-mediated granuloma formation. Indeed, anti-TB immune reaction and granuloma formation classically involve macrophages, dendritic cells, NK cells, γδ, CD4 and CD8αβT-lymphocytes. Both HIV and TB infections impair this cellular immunity which recovers after the pathogens are controlled by both antiviral and antimycobacterial therapies.

The T cell reconstitution observed after 3 to 6 months after HAART onset and successful control of HIV (Li et al. (1998) Lancet 351:1682-1686, Autran et al. (1997) Science 277:112-116) is associated to recovery of DTH (delayed type hypersensitivity reaction) reactivity to tuberculin (Wendland et al. (1999) Aids 13:1857-1862) and of tuberculin-specific T-lymphocytes. Those cells may represent up to 33% of peripheral CD4⁺T-lymphocytes in M. tuberculosis co-infected patients (Hengel et al. (2002) AIDS Res Hum Retroviruses 18:969-975), suggesting that an acute restoration of functional tuberculin-specific T-lymphocytes specific for M. tuberculosis might be involved in the pathophysiology of TB-IRS.

The brutal exacerbation of tuberculin-specific T-lymphocytes might also reveal a restoration process in antigen presenting cells such as myeloid dendritic cells (mDC) involved in the early activation of specific anti-TB Th1 immune response (Jiao et al. (2002) J Immunol 168:1294-1301.) Both HIV and M. tuberculosis are able to regulate immune response orientation by DC via a TLR-2/DC-SIGN pathway interaction balance (Geijtenbeek et al. (2003) J Exp Med 197:7-17) while HIV-infection has been associated to defects in circulating dendritic cells both myeloid and plasmacytoïd (Grassi et al. (1999) Aids 13:759-766) which are partially restored by anti-retroviral and anti-tuberculosis therapy (Lichtner et al. (2006) Clin Exp Immunol 143:329-337, Schmidt et al. (2006) J Clin Immunol 26:55-64, Zhang et al. (2006) J Immunol 176 :5644-5651).

Another T cell subset appears to play a predominant role against M. tuberculosis infection, i.e., the γδT-lymphocytes which are strong and early IFN-γ producing cells in response to mycobacterial antigens. Of particular relevance are the γδT-lymphocytes using a Vδ2 chain Those cells are quantitatively and functionally impaired by both HIV (Autran et al. (1989) Clin Exp Immunol 75:206-210) and M. tuberculosis infections (Carvalho et al. (2002) Thorax 57:357-360) with a dysbalance between the Vδ1 and the normally predominant Vδ2⁺ subsets (Boullier et al. (1995) J Immunol 154:1418-1431). In addition an anergy of the Vδ2⁺ sub-population to organophosphosphoric antigens is observed during HIV and TB infection (Rojas et al. (2005) J Infect Dis 192:1806-1814, Gioia et al. (2002) J Immunol 168:1484-1489). After HAART and anti-TB therapies, these deficiencies recover (Shen et al. (2004) J Infect Dis 190:1438-1447), except for the amplification of the Vδ1⁺ population (Poles at al. (2003) J Virol 77:10456-10467), and have already been associated to inflammatory syndromes in SIV-infected macaques (Shen et al. (2004) J Infect Dis 190:1438-1447). Moreover the γδ T cell effector function is known to be modulated by inhibitory and activatory CMH-I ligands receptors belonging to the NK Receptors. These modulations are used by pathogens to escape immune reaction: TB increases the NKG2A inhibitor expression on Vδ2⁺ cells (Boullier et al. (1998) Eur J Immunol 28:3399-3410), whereas HIV-1 increases the NKG2C activator on Vδ1⁺T-lymphocytes (Fausther-Bovendo et al. (2008) Aids 22:217-226). Such a KIR over-expression (corresponding to a CD94, CD158a,b over-expression) has also been shown on conventional CD8T-lymphocytes using an αβ TCR during HIV-1 infection that was proposed to participate in the immune deficiency (Costa et al. (2001) Aids 15:965-974).

There is a need to find a method to identify patients who are likely to develop a TB-IRS and to develop tools for positive diagnosis of TB-IRS in patients who are developing this syndrom. Particularly, there is a need to find a method to identify patients infected with M. tuberculosis or co-infected with M. tuberculosis and HIV, who are likely to develop a TB-IRS, in order to adapt the therapy accordingly. There is also a need for a method which allows a quick diagnosis of TB-IRS after the beginning of the treatment with HAART and/or with anti-TB therapy.

It was previously reported that co-infected patients under HAART treatment who develop an IRS produce antibodies against PGL-Tb1. However, this characteristic is visible only under HAART treatment and cannot be used in a predictive test (Simmoney et al. (2008) tuberculosis 88 (5): 453-61). It was also shown that the pre-treatment numbers of CD8⁺ CD25⁺ cells in patients who developed TB-IRS were higher than those in patient who did not develop TB-IRS (Cianchetta-Sivori et al. (2008) Aids 21(17):2347-9).

In order to identify predictive factors of TB-IRS, the inventors sought to identify the characteristics of the major actors of the M. tuberculosis related granulomatous responses in IRS and non-IRS patients.

### Method of Prognosing and/or Diagnosing TB-IRS

The inventors identified that patients who will develop a TB-IRS immunologically differ from those who will not develop a TB-IRS by a significantly lower proportion of γδ⁺T-lymphocytes displaying an inhibitory KIR receptor such as CD158a, CD158b or CD94 at time of treatments initiation. In particular patients who will develop a TB-IRS have a significantly lower proportion of γδ⁺ or Vδ2⁺ γδ⁺T-lymphocytes displaying an inhibitory KIR receptor. It was demonstrated these inhibitory KIR receptors acted as negative regulators of the IFN-γ production in the γδ⁺ and the γδ⁺Vδ2⁺T cells.

Furthermore, expression of inhibitory KIR receptor(s) on γδ⁺ or Vδ2⁺γδ⁺T-lymphocytes remained significantly lower in patients having developed a TB-IRS after HAART onset as compared with patients who did not develop a TB-IRS while the proportion of total Vδ2⁺ γδ⁺T-lymphocytes increased at time of TB-IRS.

In parallel the previously described increase in tuberculin-specific T cells could be assigned to tuberculin-specific CD4 T cells using an αβ TCR, which represent therefore a distinct mechanism involved in IRS. However before initiation of treatment , and in contrast to what observed for the γδ Vδ2 T cells numbers of tuberculin-specific CD4 T cells do not differ between patients who will develop TB-IRS and those who will not.

Therefore these results allow proposing a method of prognosing the development of TB-IRS in individuals. Particularly these results allow proposing a method of prognosing the development of TB-IRS in individuals infected with M. tuberculosis, or co-infected with M. tuberculosis and HIV, before initiation of anti-TB treatment and/or HAART, or who are receiving anti-TB treatment and/or HAART but do not show clinical symptoms of TB-IRS.

These results also allow proposing a method of diagnosing TB-IRS. Particularly these results allow proposing a method of diagnosing TB-IRS in individuals infected with M. tuberculosis, or co-infected with M. tuberculosis and HIV, who are receiving anti-TB treatment and/or HAART.

In both prognosing and diagnosing methods, the detection of a low percentage or concentration of γδ⁺ or γδ⁺ Vδ2⁺T-lymphocytes which express an inhibitory KIR receptor, is indicative of a risk of developing TB-IRS or is a confirmation that the individual is developing TB-IRS.

The invention thus relates to a method of prognosing and/or diagnosing a M. tuberculosis-Immune restoration syndrome (TB-IRS) in an individual infected with M. tuberculosis and optionally with HIV comprising the steps consisting of:

a) determining the percentage or concentration of γδ⁺ or γδ⁺Vδ2⁺T-lymphocytes which express an inhibitory KIR receptor in a biological sample of said individual;

b) comparing said percentage or concentration of γδ⁺ or γδ⁺Vδ2⁺T-lymphocytes expressing an inhibitory KIR receptor to a predetermined value; and

c) characterizing the individual's risk of developing a TB-IRS or determining if the individual suffers from TB-IRS based upon the comparison of said percentage or concentration of γδ⁺ or γδ⁺Vδ2⁺T-lymphocytes expressing an inhibitory KIR receptor with the predetermined value.

As used herein, “M. tuberculosis-Immune restoration syndrome” or “TB-IRS” denotes a syndrome occurring in individuals infected with M. tuberculosis, or co-infected with HIV and M. tuberculosis. Clinical symptoms include fever, lymphadenopathy, worsening of respiratory and other initial TB symptoms, and exacerbation of granulomatous lesions. TB-IRS may occur in particular in individuals receiving anti-tuberculosis treatment, anti-retroviral treatment, or both of them.

“Human Immunodeficiency Virus” or “HIV” denotes HIV-1 of any group (A-H or O), or HIV-2 of any group (A, B). Preferably HIV is HIV-1.

“Highly Active Anti Retroviral Therapy” or “HAART” is the term used by the one skilled in the art to designate a combination treatment of multiple anti-HIV drugs, or multitherapy. Different classes of antiviral agents may be combined for HAART, e.g. integrase inhibitors which include strand transfer inhibitors (INSTIs) and integrase binding inhibitors (INBIs); DNA reverse transcriptase inhibitors which include nucleoside and non-nucleoside reverse transcriptase inhibitors; protease inhibitors; entry inhibitors; fusion inhibitor; and virus maturation inhibitors.

The “percentage of γδ⁺T-lymphocytes which express an inhibitory KIR receptor” refers to the proportion γδ⁺T-lymphocytes which express at least one type of inhibitory KIR receptor within the total population of γδ⁺T-lymphocytes in a biological sample of the individual.

The “percentage of γδ⁺ Vδ2⁺T-lymphocytes which express an inhibitory KIR receptor” refers to the proportion of γδ⁺ Vδ2⁺T-lymphocytes which express at least one type of inhibitory KIR receptor within either the total population of γδ⁺T-lymphocytes or the total subpopulation of γδ⁺ Vδ2⁺T-lymphocytes in a biological sample of the individual.

As used herein, the “concentration” of γδ⁺ or γδ⁺ Vδ2⁺T-lymphocytes expressing an inhibitory KIR receptor refers to the number of such T-lymphocytes in the biological sample.

γδ⁺ Vδ2⁺T-lymphocytes denote CD3⁺T-lymphocytes with a TCR composed of a gamma chain and a Vδ2 delta chain. The γδ⁺ Vδ2⁺T-lymphocytes are the sub-population of γδ⁺T-lymphocytes which recognize organophosphoric antigens from mycobacteria.

As used herein the term “KIR” denotes “Killer Immunoglobulin-like Receptor”, and by extent more generally “Killer Inhibitory Receptor”. In the content of invention, KIR includes receptor members of the CD158 family, receptor members of the CD94/NKG2 family, and NKG2D receptor.

The CD158 family consists of Killer Immunoglobulin-like Receptor proteins which possess characteristic Ig-like domains on their extracellular regions, which in some of these KIR proteins are involved in HLA class I ligand binding. Receptors of the CD94/NKG2 family are type II, C-type, lectin-like membrane glycoproteins which are expressed on the cell surface as heterodimers with CD94. NKG2D (also called KLR-G1) was initially placed in the CD94/NKG2 family but it does not form heterodimers with CD94.

KIR of the CD158 and CD94/NKG2 families may have inhibitory or activating activity on NKT cells and CTL. Inhibitory KIRs are receptors which, when expressed on NKT cells or CTL and upon interaction with a class I HLA molecules (in particular HLA-C molecules) negatively regulate the cytotoxicity of the NKT cell or CTL.

Inhibitory KIR receptors of the CD158 family comprise KIR2DL1 (also called CD158a), KIR2DL2 (also called CD158b1), KIR2DL3 (also called CD158b2), KIR3DL1 (also called CD158e1), KIR3DL2 (also called CD158k), KIR2DL4, and KIR2DL5. As used herein, “CD158b” refers to CD158b1 or CD158b2.

Inhibitory KIR receptors of the CD94/NKG2 family are preferably CD94/NKG2A here also referred to as CD94. NKG2D receptor is also an inhibitory KIR receptor which may be expressed by NKT and CTL.

These families of receptors are described in more details in Iannello et al. (2008, Journal of Leukocytes Biology 84:1-49).

Preferably, the inhibitory KIR receptor is selected from the group consisting of CD158b1 and CD158b2, CD94/NKG2A and CD158a.

According to the method of the invention, in step a) a percentage or concentration of γδ⁺ or γδ⁺Vδ2⁺T-lymphocytes which express one type of inhibitory KIR receptor may be determined. In other words, the method may involve for instance determining the percentage or concentration of γδ⁺ or γδ⁺Vδ2⁺T-lymphocytes which express CD94/NKG2A, or the percentage or concentration of γδ⁺ or γδ⁺Vδ2⁺T-lymphocytes which express CD158a, or the percentage or concentration of γδ⁺ or γδ⁺Vδ2⁺T-lymphocytes which express CD158b, etc., as compared with the total population of γδ⁺ or γδ⁺Vδ2⁺T-lymphocytes in the biological sample of the individual.

The method of the invention may also involve determining a percentage or a concentration of γδ⁺ or γδ⁺Vδ2⁺T-lymphocytes which express a combination of two or more types of inhibitory KIR receptors, as compared with the total population of γδ⁺ or γδ⁺Vδ2⁺T-lymphocytes in the biological sample of the individual. For instance, the percentage or concentration of γδ⁺ or γδ⁺Vδ2⁺T-lymphocytes which express CD158a and CD158b may be determined, or the percentage or concentration of γδ⁺ or γδ⁺Vδ2⁺T-lymphocytes which express CD94/NKG2A and CD158a, or the percentage or concentration of γδ⁺ or γδ⁺Vδ2⁺T-lymphocytes which express CD94/NKG2A and CD158b, or the percentage or concentration of γδ⁺ or γδ⁺Vδ2⁺T-lymphocytes which express CD94/NKG2A, CD158a and CD158b, etc.

The method of the invention involves comparing the percentage or concentration of γδ⁺ or γδ⁺ Vδ2⁺T-lymphocytes which express an inhibitory KIR receptor with a predetermined value. The predetermined value can take a variety of forms.

The predetermined value can be a single value, such as percentage or a concentration of γδ⁺ Vδ2⁺T-lymphocytes which express said inhibitory KIR receptor(s) as determined in a reference group of individuals infected with M. tuberculosis, and optionally infected with HIV, who did not develop TB-IRS upon treatment with anti-TB and/or HAART. The one skilled in the art will readily understand that if in step a) the percentage or concentration of γδ⁺ or γδ⁺Vδ2⁺T-lymphocytes which express CD158a was determined, then the predetermined value would be respectively a percentage or a concentration of γδ⁺ or γδ⁺Vδ2⁺T-lymphocytes which express said CD158a in a reference group of individuals. Similarly, if in step a) the percentage or concentration of γδ⁺ or γδ⁺Vδ2⁺T-lymphocytes which express CD158a and CD158b was determined, then the predetermined value would be respectively a percentage or a concentration of γδ⁺ or γδ⁺Vδ2⁺T-lymphocytes which express said CD158a and CD158b in a reference group of individuals; etc.

Said single predetermined value may be for instance the median, the mean percentage, the mean concentration, or the median thereof of γδ⁺ or γδ⁺Vδ2⁺T-lymphocytes which express said inhibitory KIR receptor(s).

When the predetermined value is a single value, then a percentage or concentration of γδ⁺ or γδ⁺ Vδ2⁺T-lymphocytes expressing said inhibitory KIR receptor(s) lower than the predetermined value is indicative of a risk of developing TB-IRS or is indicative that the individual suffers from TB-IRS. In particular, the percentage or the concentration of γδ⁺ or γδ⁺Vδ2⁺T-lymphocytes expressing said inhibitory KIR receptor(s) measured in the biological sample of the individual may be at least 10%, 20%, 30%, 40%, 50%, 60%, 70%, 80%, 90%, 100%, 150% or 200% lower than the predetermined value.

The predetermined value may also be established based upon comparative groups, such as where the risk in one defined group is double the risk in another defined group. The predetermined value can be a range, for example, where the tested population is divided equally (or unequally) into groups, such as a low-risk group, a medium-risk group and a high-risk group, or into quantiles, the lowest quantile (i.e. the quantile gathering individuals having the lowest percentage or concentration of γδ⁺ or γδ⁺Vδ2⁺T-lymphocytes expressing said inhibitory KIR receptor(s)) being individuals with the highest risk and the highest quantile (i.e. the quantile gathering individuals having the highest percentage or concentration of γδ⁺ or γδ⁺Vδ2⁺T-lymphocytes expressing said inhibitory KIR receptor(s)) being individuals with the lowest risk.

Accordingly, in the method of the invention, the predetermined value may comprise multiple ranges of percentage or concentration of γδ⁺ or γδ⁺Vδ2⁺T-lymphocytes expressing said inhibitory KIR receptor(s) measured in a population of individuals infected with M. tuberculosis, and optionally with HIV, said population being divided into quantiles according to the percentage or concentration of γδ⁺ or γδ⁺Vδ2⁺T-lymphocytes expressing said inhibitory KIR receptor(s), the lowest quantile(s) being individuals with the highest risk of developing TB-IRS or individuals having TB-IRS. For instance, the population of individuals infected with M. tuberculosis, and optionally with HIV could be divided into 2, 3, 4, or 5 quantiles. Individuals in the lowest quantile, or the two lowest quantiles (if the population is divided e.g. into 4, or 5 quantiles) would be considered as individuals at risk of developing IRS or as individuals likely having TB-IRS.

The individual infected with M. tuberculosis, or co-infected with M. tuberculosis and HIV, may or may not be receiving a treatment against M. tuberculosis and/or against HIV at the time the method according to the invention is performed. According to an embodiment, said co-infected individual has received an anti-tuberculosis treatment before onset of HAART, and the anti-tuberculosis treatment is preferably continued after HAART is initiated. According to another embodiment, said co-infected individual has not received any anti-tuberculosis treatment before onset of HAART, and anti-tuberculosis treatment is initiated at the same time as HAART, or shortly after, i.e. at the time the method according to the invention is performed the individual is receiving HAART and anti-tuberculosis treatment.

The method according to the invention may be performed in a biological sample obtained from an individual infected with M. tuberculosis, or co-infected with M. tuberculosis and HIV, before onset of anti-TB and HAART treatment. The method is then a method of prognosing TB-IRS.

The method may also be performed in a biological sample obtained from an individual infected with M. tuberculosis, or co-infected with M. tuberculosis and HIV, who is undergoing anti-TB treatment and/or HAART. In particular, the method may be carried out in a biological sample obtained from an individual who does not exhibit clinical symptoms of TB-IRS, in order to determine if the individual is likely to develop TB-IRS. The method may also be performed in a biological sample obtained from an individual infected with M. tuberculosis, or co-infected with M. tuberculosis and HIV, undergoing anti-TB and/or HAART who is suspected to have TB-IRS.

The inventors have also observed that a significant expansion of γδ⁺ or γδ⁺Vδ2⁺T-lymphocytes, in particular circulating γδ⁺ or γδ⁺Vδ2⁺T-lymphocytes, is associated with the occurrence of TB-IRS. Accordingly, monitoring an increase in the percentage or concentration of γδ⁺ or γδ⁺Vδ2⁺T-lymphocytes within the population of γδ⁺T-lymphocytes, in the course of anti-TB treatment and/or HAART, is an additional mean enabling to establish the prognosis of TB-IRS.

Accordingly, the method of the invention may further comprise monitoring the percentage or concentration of γδ⁺ or γδ⁺Vδ2⁺T-lymphocytes before initiation and/or during anti-TB and/or of HAART treatment, wherein an increased percentage or concentration of γδ⁺ or γδ⁺Vδ2⁺T-lymphocytes in the course of treatment is indicative of a risk of TB-IRS or of having TB-IRS. The monitoring is typically performed by determining the percentage or concentration of γδ⁺ or γδ⁺Vδ2⁺T-lymphocytes expressing inhibitory KIR(s) at different points in time during the treatment, for instance at intervals of 2 weeks, 1 month, 2 months, 3 months, etc. In particular, said method may comprise determining a percentage or concentration of γδ⁺ or γδ⁺Vδ2⁺T-lymphocytes expressing an inhibitory KIR before and after onset of treatment, wherein an increased percentage or concentration of γδ⁺ or γδ⁺Vδ2⁺T-lymphocytes expressing an inhibitory KIR receptor after onset of treatment as compared with the percentage or concentration of γδ⁺ or γδ⁺Vδ2⁺T-lymphocytes expressing an inhibitory KIR receptor before onset of treatment is indicative of a risk of TB-IRS or of having TB-IRS.

The international patent application WO 2007/039400 previously described that TB-IRS can be diagnosed by detecting an acute increase in Th1 Lymphocytes response following exposure to M. tuberculosis antigen PPD (Purified Protein Derivative) and/or 16 kDa protein, but not to ESAT-6 or CFP-10 antigens from M. tuberculosis. The inventors have found these Th1 INF-γ producing cells were CD4+ TCRαβ T cells. Therefore, the method of diagnosing according to the invention may further comprise monitoring the level of CD4⁺ lymphocytes which are specific for M. tuberculosis PPD and/or 16 kDa protein in the course of HAART, wherein an increase in the level of said CD4⁺ lymphocytes, preferably at least by 250%, is indicative of TB-IRS.

According to the invention, the biological sample may be blood or a tissue sample or another biological liquid. Preferably, the biological sample is a biological liquid selected from the group consisting of whole blood, or blood fractions such as Peripheral Blood Mononuclear Cells (PBMCs), or of broncho-alveolar lavage (BAL) liquid, cerebrospinal fluid (CSF) ascitic fluid, pleural fluid, or tissue mononuclear cells which may be obtained by needle aspiration or biopsy.

### Methods of Determining the Percentage of T-lymphocytes Subpopulations

The percentage or concentration of γδ⁺ or γδ⁺Vδ2⁺T-Lymphocytes expressing an inhibitory KIR receptor can be carried out by any suitable method known in the art.

Antibody-based techniques, such as flow cytometry or immuno-enzymatic technics may generally be used to readily determine the pattern of inhibitory KIR(s) expression at the surface of γδ⁺ or γδ⁺Vδ2⁺T-Lymphocytes, in particular where the percentage of γδ⁺ or γδ⁺Vδ2⁺T-Lymphocytes expressing a combination of inhibitory KIR receptors is to be determined. These methods typically involve the use of at least one antibody specific for a given inhibitory KIR receptor which is directly or indirectly labelled with a fluorescent dye. If a combination of inhibitory KIR receptors is to be determined, several antibodies, each of them being specific for a given inhibitory KIR receptor, may be used. In such a case, the antibodies may be advantageously labelled with fluorescent dyes having distinguishable emission wavelength.

For instance the sub-population γδ⁺ or γδ⁺Vδ2⁺T-Lymphocytes may be characterized with anti-TCRδ2-FITC and at least one antibody directed against a KIR receptor, such as CD94, CD158a, CD158b or NKG2D, labelled with a different fluorescent dye, such as phycoerythrin (PE). If several KIR receptor antibodies are to be used, suitable fluorescent dyes may be readily selected by the one skilled in the art depending on the excitation wavelength available with the flow cytometry analyser to be used. Fluorescent dyes include for instance FITC (Fluorescein isothiocyanate), R-PE (R-Phycoerythrin), PE-Texas Red, PE-Cy5, PE-Cy5.5, PerCP-Cy5.5, PE-Cy7 or any fluorochrome to be used in flow cytometry or cell imaging systems.

More specifically, the pattern of inhibitory KIR(s) expression in the population of γδ⁺ or γδ⁺Vδ2⁺T-Lymphocytes may be established at the transcriptional level, by detecting inhibitory KIR mRNA(s), or at the traductional level, by detecting expression of inhibitory KIR protein(s) on γδ⁺ or γδ⁺Vδ2⁺T-Lymphocytes.

For instance, the percentage or concentration of the subpopulation of γδ⁺ or γδ⁺Vδ2⁺T-Lymphocytes among γδ⁺T-Lymphocytes may be quantified by flow cytometry, while sorting γδ⁺ or γδ⁺Vδ2⁺T-Lymphocytes at the same time. Then, in the isolated subpopulation of γδ⁺ or γδ⁺Vδ2⁺T-Lymphocytes, the pattern of inhibitory KIR(s) expression may be established using probes and/or primers specific for the inhibitory KIR mRNA(s) in order to quantify said mRNAs by hybridization and/or amplification techniques. The sequences of inhibitory KIR receptors are publicly available and the one skilled in the art would readily know how to design probes and/or primers specific for a given KIR mRNA.

### Kits

The invention also relates to a kit for prognosing and/or diagnosing M. tuberculosis-Immune restoration syndrome (TB-IRS) in an individual infected with M. tuberculosis or co-infected with HIV and M. tuberculosis, which comprises an antibody directed against TCRδ2 and at least one antibody directed against an inhibitory KIR receptor. Preferably, said kit comprises instructions for use in prognosing and/or diagnosing TB-IRS.

In particular, said at least one antibody directed against an inhibitory KIR receptor may be selected from the group consisting of an antibody directed against CD94, an antibody directed against CD158a, an antibody directed against CD158b, and an antibody directed against NKG2D receptor.

The invention will be further illustrated in view of the following figures and examples.

### FIGURES

FIG. 1 depicts the surface expression of the Ig-like inhibitor Receptor CD158a on CD3⁺/⁻ γδ⁺/⁻ or Vδ2⁺/⁻T-lymphocytes in 11 IRS (in black) and 13 non-IRS (in white) patients under antimycobacterial therapy when initiating HAART (M₀).

FIG. 2 depicts the surface expression of the Ig-like inhibitor Receptor CD158b on CD3⁺/⁻ γδ⁺/⁻ or Vδ2⁺/⁻T-lymphocytes in 11 IRS (in black) and 13 non-IRS (in white) patients under antimycobacterial therapy when initiating HAART (M₀).

FIG. 3 depicts the surface expression of the lectin-like heterodimer CD94/NKG2 on Vδ2⁺/⁻ T, CD3⁺/⁻ γδ⁺/⁻ or CD3⁺/⁻ cells in 11 IRS (in black) and 13 non-IRS (in white) patients under antimycobacterial therapy when initiating HAART (M₀).

FIG. 4 depicts the percentage of CD94⁺/TCR gamma delta cells in IRS patients (in black) and non-IRS patients (white) when initiating antimycobacterial therapy (TBK), when initiating HAART (M₀), at 1 month after HAART initiation or at IRS time (M₁/TIRS), at 3 months after HAART initiation (M₃), at 6 months after HAART initiation (M₆), at 12 months after HAART initiation (M₁₂).

FIG. 5 depicts the effect of stimulation with anti-CD3 in one IRS patient on the production of INF-γ in Vδ2⁺ cells or Vδ2⁻ cells, CD94⁻, CD158a⁻ or CD158b⁻ cells compared to Vδ2⁺cells or Vδ2⁻cells, CD94⁺, CD158a⁺ or CD158b1⁺.

FIG. 6 depicts the FACS analysis of T-lymphocytes producing INF-γ and presenting the CD4⁺ marker or the HLA-DR complex in one patient treated with antimycobacterial therapy when initiating HAART (M₀).

FIG. 7 depicts the FACS analysis of T-lymphocytes producing INF-γ and presenting the CD4 marker in five patients at time of IRS (TIRS).

FIG. 8 depicts the differentiation profile of tuberculin-specific CD4⁺ T lymphocytes estimated by the expression of HLADR, CD45RA, CD27, INFγ, TNFα measured by FACS in one IRS patient at time of IRS (TIRS).

FIG. 9 depicts the median percentage of CD4⁺ or CD8⁺T-lymphocytes of CD4⁺ HLADR⁺/CD3⁺ cells (triangles) CD8+ HLADR⁺/CD3⁺ cells (squares) in 11 IRS (black) or 13 non-IRS (white) patients when initiating antimycobacterial therapy (TBK), HAART (M₀), at 1 month after HAART initiation or IRS time (M₁/TIRS) and at 3 (M₃), 6 (M₆), 12 (M₁₂) months after HAART initiation.

FIG. 10 depicts the median percentage of TCR γδ⁺/CD3⁺ (squares) and of HLADR⁺/γδ⁺/CD3⁺T-lymphocytes (triangles) in 11 IRS (black) or 13 non-IRS (white) patients when initiating antimycobacterial therapy (TBK), HAART (M₀), at 1 month after HAART initiation or IRS time (M1/TIRS) and at 3 (M₃), 6 (M₆), 12 (M₁₂) months after HAART initiation.

FIG. 11 depicts the median percentage of CD3⁺ TCR γδ⁺T-lymphocytes of Vδ2-γδ T-lymphocyte (squares) and Vδ2⁺-γδT-lymphocytes (triangles) in 11 IRS (black) or 13 non-IRS (white) patients when initiating antimycobacterial therapy (TBK), HAART (M₀), at 1month after HAART initiation or IRS time (M₁/TIRS) and at 3 (M₃), 6 (M₆), 12 (M₁₂) months after HAART initiation.

### EXAMPLES

### Example 1

### Patients Included in the Study

### Methods

Thirty-five consecutive TB-HIV co-infected untreated patients were prospectively included when beginning anti-TB treatment. Inclusion criteria were: HIV-1 infection, no previous HAART, CD4 count below 200 cells/mm³, anti-TB therapy initiated and indication to further HAART initiation. Inclusion was secondly confirmed when M. tuberculosis infection was proven (positive culture or histological findings). Patients were evaluated when initiating antimycobacterial therapy (T_{BK}), HAART (M₀), and at 1, 3, 6 and 12 months after HAART initiation (M_{1,3,6,12}). In addition, patients with IRS (IRS) were evaluated at IRS time (T_{IRS}) and 20 days later. IRS was defined as follows (French et al. (2004) AIDS 15:965-974): recurrence of inflammatory reaction (fever, elevated CRP), enlargement of pre-existing lesions, or development of new lesions (lymph nodes, pleuritis) with no mycobacterium resistance, no other diagnosis and with a response to HAART (HIV-RNA decrease >1 log copies/ml). Patients who did not experience IRS within three months after HAART initiation were defined as NON-IRS. Eleven patients initially included were excluded: 4 did not confirm TB infection, 4 changed of medical centre, 2 were lost in follow-up within three months of HAART onset and one was transferred early in ICU for seizure. Therefore the immunological investigations were performed on the 24 analysable patients.

The study was accepted by the Saint-Louis Hospital Institutional-Ethical-Committee and all patients signed an informed consent.

### Results

The prospectively analysis showed that eleven (46%) of the 24 patients developed IRS symptoms in a median of 23 days [range 7-85] after HAART onset. As shown in Table 1, IRS and non-IRS patients did not differ neither for age, sex ratio, country of birth, HIV infection status: baseline (T_{BK} and M₀) CD4 count and HIV-1 viral load, nor for TB-infection: clinical presentation, smear-positive patients, resistance to anti-TB treatment. At time of IRS vs. M₁ for non-IRS patients, quantitative CD4 T-cells restoration and viral control were identical in both groups.

TABLE 1
Clinical characteristics of IRS and non-IRS groups

| | | IRS | | non-IRS | | p |
|---|---|---|---|---|---|---|
| | n | 11 | | 13 | | |
| | Gender (M/F) | 6/5 | | 9/4 | | NS |
| | Age (yr) | 41 | (30-56) | 37 | (26-63) | NS |
| TB | Pulmonary TB | 2/11 | | 4/13 | | NS |
| infection | | | | | | |
| | Disseminated TB | 9/11 | | 9/13 | | NS |
| | Smear positive | 5/11 | | 4/13 | | NS |
| HIV | CD4 (/mm³) | 37 | (3-123) | 56 | (13-330) | NS |
| infection (M₀) | | | | | | |
| | VL (log) | 5.7 | (4.6-6.5) | 5.2 | (4.3-5.9) | NS |
| M_{0HAART} | days from T_{BK} | 36 | (7-77) | 50 | (14-111) | NS |
| TIRS or | days from | 23 | (2-82) | | | |
| M₁ (non-IRS | M_{0HAART} | | | | | |
| patients) | | | | | | |
| | CD4 (/mm³) | 108 | (59-430) | 163 | (9-580) | NS |
| | ΔCD4 from M₀ | +54 | (−1; +393) | +77 | (−50; +250) | NS |
| M₃ | CD4 (/mm³) | 117 | (58-399) | 132 | (49-410) | NS |
| | ΔCD4 from M₀ | +86 | (−74; +367) | +73 | (−88; +354) | NS |

| | | | |
|---|---|---|---|
| VL < 200 cp/ml (n) | 7/10 | 8/11 | NS |

Medians (Range); VL, viral load, Mann Withney non parametric tests between IRS and non-IRS patients.

### Example 2

### Analysis Before and After Onset of HAART of the Characteristics of the Major Actors of the M. tuberculosis Related Responses in IRS and non-IRS Patients

### Methods

### ELISPOT Assay for Quantification of Mycobacterial-Specific Th1 Cells

Antigen-specific Th1 cells producing IFN-γ were prospectively quantified on fresh peripheral blood mononuclear cells (PBMC) by ELISpot as described (Bourgarit et al. (2006) Aids 20:F1-7), after a 40-hour stimulation with mycobacterial extracts (Tuberculin, 1 μg/ml; Statens Serum Institute, Copenhagen, Denmark). Controls were phytohaemagglutinin (Murex) and medium alone. Spots were counted using an ELISpot reader (Zeiss) and data were expressed as spot-forming-cells (SFC)/10⁶PBMCs. Results were considered as positive if above a 50 SFC/10⁶ PBMC after substraction of the mean background obtained with cells alone.

### Membrane Phenotyping

The phenotype of peripheral blood cells was analysed on fresh whole blood by four-colour flow cytometry using standard methods (Li et al. (1998) Lancet 351:1682-1686, Autran et al. (1997) Science 277:112-116). Stained cells were analysed on a FACScalibur (Becton-Dickinson, “BD” hereafter) as described. The different circulating populations were defined and characterized as follows.

αβ T-cells were stained with the following antibody combinations: anti-HLA-DR-FITC (Immunotech), anti-CD25-PE (BD), anti-CD4-PerCPCy5 (BD) or anti-CD8-PerCP (BD) and anti-CD3-APC (BD), anti-45RA-FITC (Immunotech) anti-62L-PE (pharmingen), anti-CD7-PE (Coulter clone).

Gamma-Delta (γδ) T-lymphocytes were defined as CD3⁺ lymphocytes with pan-γδ TCR FITC or PC5 (Immunotech). Their sub-populations were characterized by anti-TCRδ2-FITC (Immunotech) and their function by analyzing HLA-DR expression (APC, BD; PeCy5 BD), NKR CD94-PE (HP-3B1, Immunotech), CD158a-PE (Coulter) and CD158b-PE (Immunotech) membrane expression. In one patient, it was possible to have the kinetic on fresh blood of the functional sub-populations characterized by CD45RA-CyC5 (Coulter) and CD27-PE (BD).

Flow cytometric analyses were performed on a FACScalibur™ with CellQuest™ Pro software (BD Biosciences) on at least 100 events for the LTVδ2 populations and on a mean of 10 000 events for total lymphocytes.

### Intracytoplasmic Cytokine Staining

αβT-lymphocytes: Frozen PBMC with viability above 80% after thawing were cultured 4 hours in RPMI medium 5%FCS. Cells were then washed and 1×10⁶ cells were stimulated for 2 hours with Tuberculin (10 μg/ml) in a 24-well flat bottom plate. Cells stimulated with medium alone and PHA (1 μg/mL) were used as negative and positive controls, respectively.

Brefeldin A was added after 2 hours and the incubation was pursued for another 18 h. After washings, cells were incubated for 15 min with the following fluorescent labeled conjugated monoclonal antibodies: anti-CD27-FITC, (BD Biosciences, France), anti-CD25 FITC, anti-CD8-FITC, anti-CD45RA-PE (Beckman-Immunotech), anti-HLA-DR-PE (BD Biosciences, France), anti-TNF-α-PE, anti-CD4-PECy5 (BD), anti-CD8-PercP (BD). After washings, cells were fixed with PFA 4%, washed in PBS-0.5% BSA -0.1% saponin buffer and then stained for 15 min at room temperature with APC-conjugated IFNγ antibody (BD Biosciences). Flow cytometric analyses were performed on a FACScalibur™ with CellQuest™ Pro software (BD Biosciences) on approximately 500,000 events.

γδ-T-lymphocytes: Frozen PBMC were stimulated for 4 hours with coated anti-CD3 (5 μg/ml) in a 24-well flat bottom plate. Cells stimulated with medium alone were used as negative controls.

Brefeldin A was added. Staining was performed as above with the following fluorescent labeled conjugated monoclonal antibodies: anti-TCRVδ2-FITC (BD Biosciences, France), anti-CD94-PE, anti-CD158a-PE ((BD Biosciences, France), anti-CD158b-PE (Beckman-Immunotech), anti-TCRγδ-biotine (e-bioscience), anti-streptavidine-PC7 (e-Bioscience) then with APC-conjugated IFNγ antibody (BD Biosciences, France).

### Statistical Analysis

Comparisons between groups were made using non-parametric tests (Fischer exact test and Mann-Whitney test) as appropriate at baseline, M₁/TIRS, and at data's peak or nadir after treatment onset. Kinetic analysis was performed by general linear model.

### Results

IRS patients have a lower proportion of γδ⁻ and Vγδ⁺T-lymphocytes displaying the inhibitor receptors CD94 and CD158a,b1.

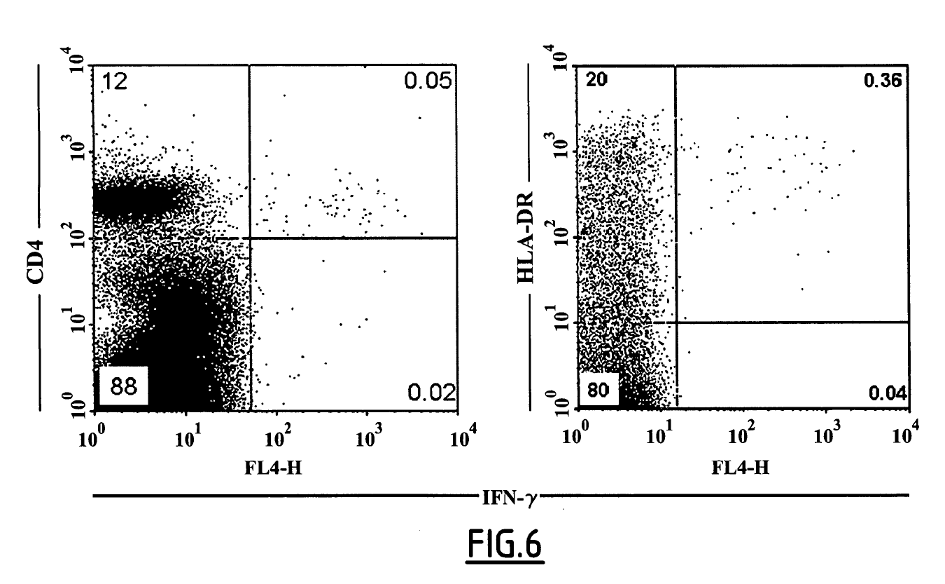

Conventional T-lymphocytes were first investigated and similar levels of tuberculin-specific IFN-γT-lymphocytes were detected in both groups with a median of 52 [18;182] SFC/10⁶ PBMC. Intracellular staining showed those cells were CD4⁺ (FIG. 6). In addition no difference was observed in activation (HLA-DR) or differentiation markers (CD45RA and CD62L) on CD4⁺ and CD8⁺T-lymphocytes (Table 2).

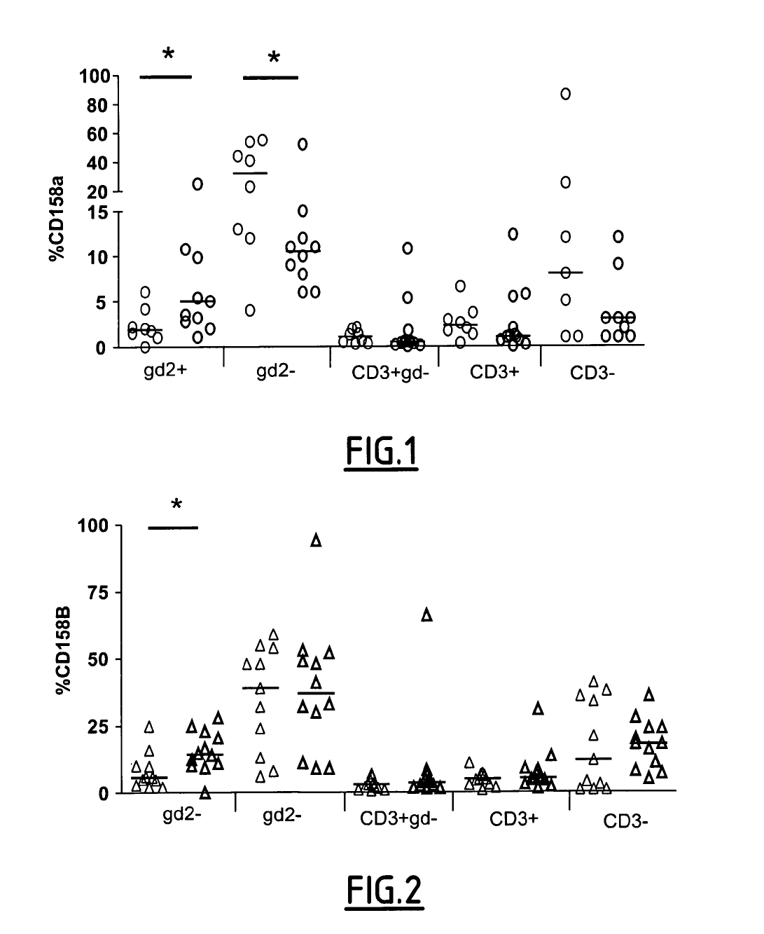

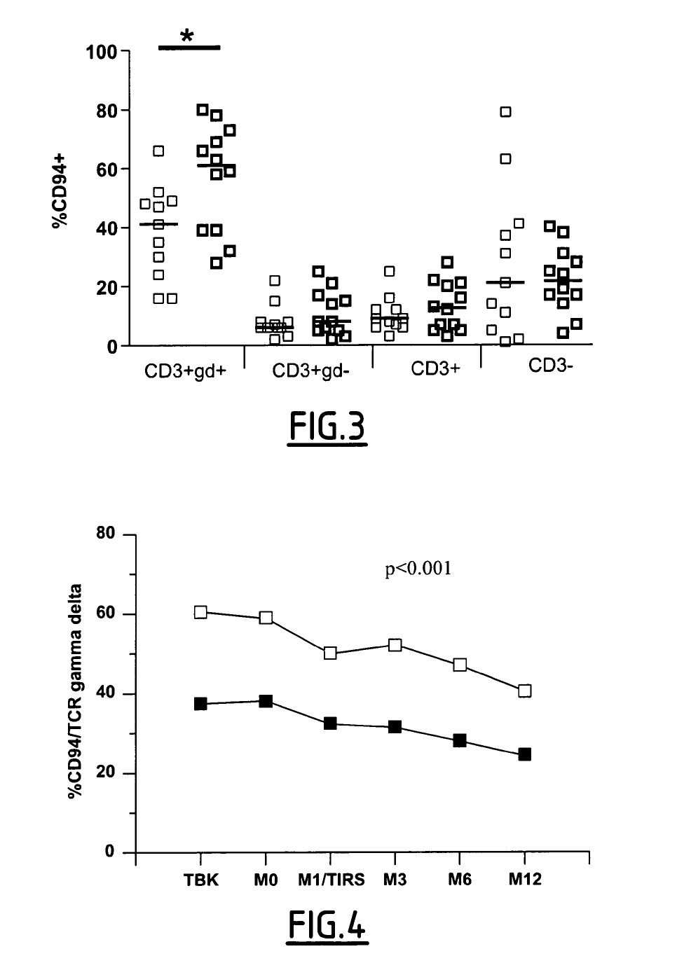

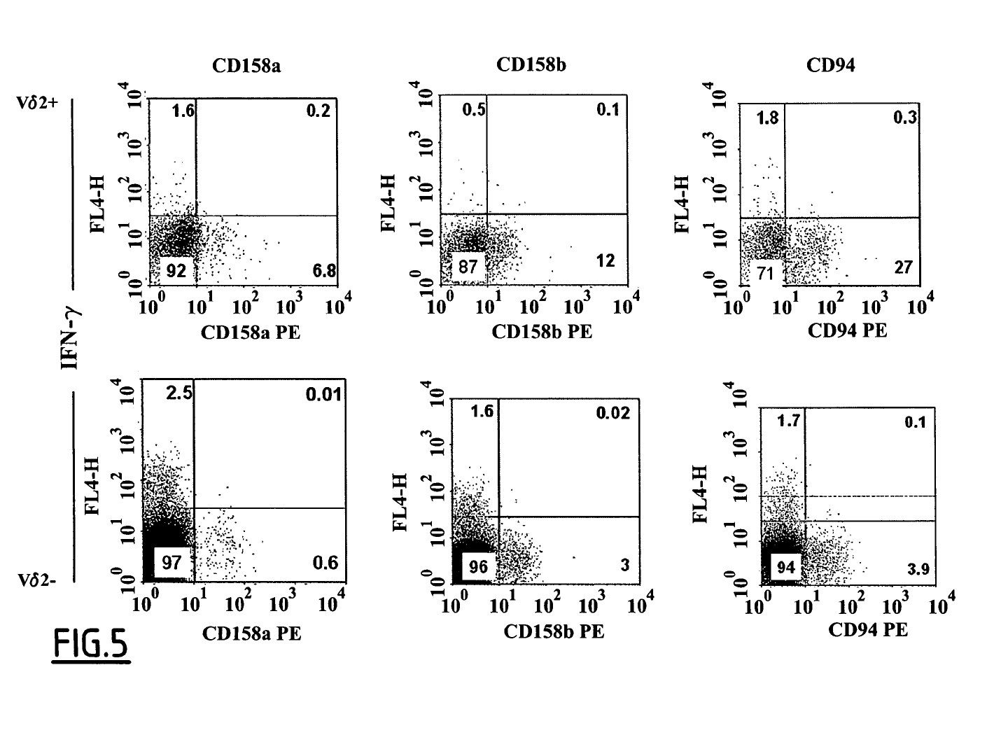

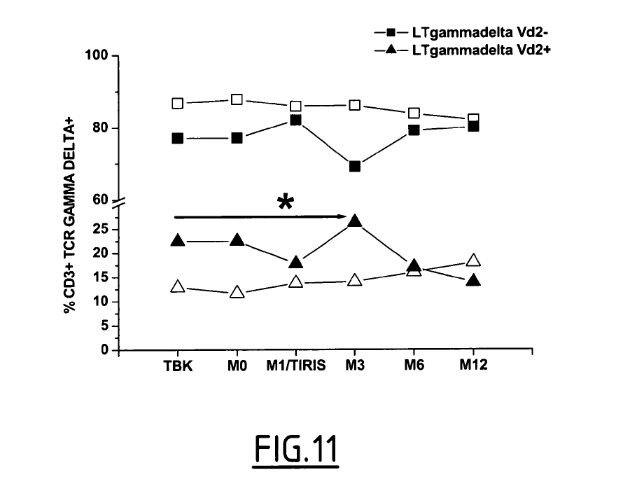

Finally, TCRγδ⁺ peripheral T-lymphocytes represented a median of 5.6% [1.5;14.4] of the CD3⁺ cells at baseline in the whole study group and those proportions did not differ between IRS patients and others. The HLA-DR activation marker expression did not differ between those two groups. As expected, the balance between the Vδ2⁺ and Vδ2⁻ γδ⁺T-lymphocytes was inverted with more Vδ2⁻ T-cells than Vδ2⁺. However, higher proportions of Vδ2⁺ cells were observed in patients who will develop IRS than the one who will not 23% [11;42] of γδ⁺T-lymphocytes vs. 12% [9;24] respectively (p=0.02, general linear model) (Table 2, FIG. 10 and FIG. 11). Surface expression of the lectin-like heterodimer CD94/NKG2 and the Ig-like inhibitor Receptors (KIRs) CD158a/KIR2DL1 (p58.1) and 158b/KIR2DL2 (p58.2) was measured on all subsets of CD3^{+/-} γδ^{+/-} and Vδ2^{+/-} cells. The CD158a and b1 expression on γδ⁺CD3⁺T-lymphocytes did not differ between IRS and others patients as on γδ⁻CD3⁺ or CD3⁻ cells. In contrast, patient's who will develop IRS displayed significantly lower frequencies of the Vδ2⁺T-lymphocytes positive for CD158a and 158b1 (p=0.03) than the one who will not (FIG. 1 and FIG. 2). Likewise, CD94⁺ T γδ⁺ cells were less frequent in IRS patients (38% [14;64] vs 63% [41;89] for non-IRS, p<0.05) (Table 2) (FIG. 3 and FIG. 4), similarly for the CD94^{brighT-lymphocytes}. The negative signalling of CD94 and CD158 was shown by the stronger IFN-γ production induced by CD3 triggering in CD94⁻ or CD158⁻ cells compared to CD94⁺ or CD158⁺ cells (FIG. 5).

In conclusion, at baseline an immunological difference between patients that will develop an IRS and those who will not is lower proportions of γδ⁺T-lymphocytes displaying inhibitory receptors CD158 and CD94 and particularly amongst Vδ2⁺ γδ⁺T-lymphocytes involved against M. tuberculosis. On the contrary, those groups did not differ for the conventional CD4⁺ Th1 cells specific for tuberculin.

TABLE 2
Baseline (M0) whole blood phenotype of T-cells, dentritic
cells (DC) and monocyte- in 11 IRS and 13 non-IRS patients

| | | IRS | | non-IRS | | p |
|---|---|---|---|---|---|---|
| LTαβCD4⁺ | % CD4⁺/CD3⁺ | 5 | [2; 11] | 9 | [3; 10] | NS |
| | % CD45RA⁻CD62L⁺ | 66 | [51; 69] | 60 | [52; 66] | NS |
| | % CD45RA⁻CD62L⁻ | 25 | [15; 38] | 25 | [14; 42] | NS |
| | % CD45RA⁺CD62L⁻ | 2 | [0; 8] | 0 | [0; 1] | NS |
| | % CD4⁺CD7⁻ | 17 | [11; 23] | 14 | [8; 19] | NS |
| | % CD4⁺HLADR⁺ | 44 | [27; 73] | 42 | [27; 52] | NS |
| | % CD4⁺CD25⁺ | 37 | [21; 48] | 43 | [27; 59] | 0.02* |
| | % CD4⁺CD25hi | 0.16 | [0; 1] | 0.4 | [0.1; 3.7] | NS |
| LTαβCD8⁺ | % CD8⁺/CD3⁺ | 83 | [77; 86] | 81 | [79; 88] | NS |
| | % CD45RA⁻CD62L⁺ | 25 | [13; 38] | 24 | [16; 30] | NS |
| | % CD45RA⁻CD62L⁻ | 36 | [20; 44] | 45 | [32; 51] | NS |
| | % CD45RA⁺CD62L⁻ | 13 | [4; 21] | 21 | [14; 28] | NS |
| | % CD8⁺HLADR⁺ | 57 | [35; 66] | 62 | [49; 75] | NS |
| LT γδ⁺ | % TCRγδ⁺/CD3⁺ | 7.3 | [3; 9] | 5.6 | [5; 7] | NS |
| | % Vδ2/γδ⁺ | 23 | [11; 42] | 12 | [9; 24] | 0.02* |
| | % HLA DR⁺/γδ⁺ | 29 | [8; 37] | 26 | [13; 40] | NS |
| | % CD94/γδ⁺ | 42 | [24; 49] | 61 | [39; 71] | 0.02 |
| | % CD158a/VVδ2⁺ | 2 | [1; 3] | 5 | [3; 11] | 0.03 |
| | % CD158b1/VVδ2⁺ | 6 | [3; 10] | 14 | [11; 22] | 0.03 |

Medians [Q1; Q3]; MannWhithey non-parametric test
*general linear model.
MFI mean fluorescence intensity.

IRS is associated with expansion of activated effector memory CD4T-lymphocytes specific for mycobacteria and of Vδ2⁺ cells.

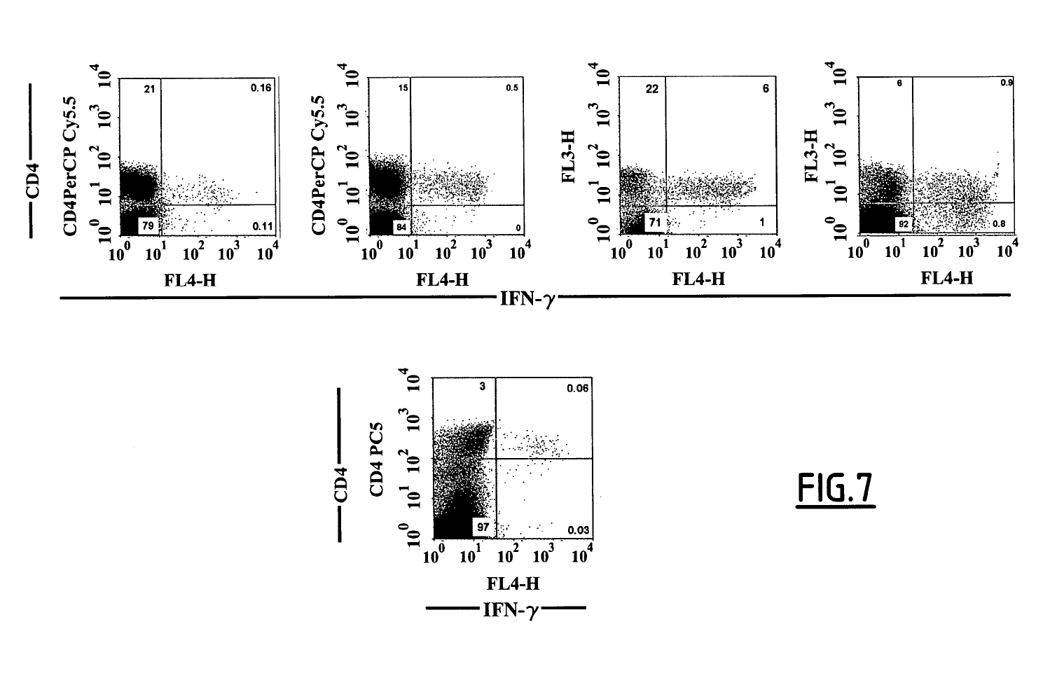

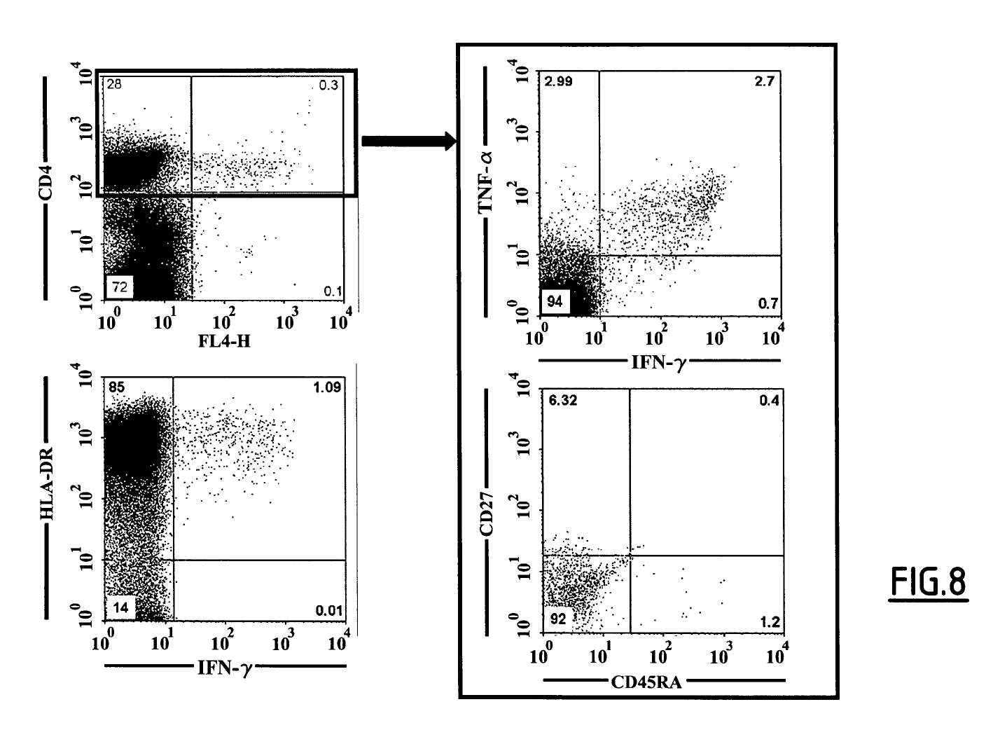

The TB-associated IRS is associated with a peak in IFN-γ producing tuberculin-specific cells reaching median values of 3,462 [1,422;3,562] SFC/10⁶ PBMC versus 453 [120;1,676] in non-IRS patients (p<0.005) as measured by ELISPOT. The nature of these IFN-γ producing cells in response to tuberculin was investigated by performing intracellular staining. At time of IRS, IFN-γ⁺T-lymphocytes represented a median of 0.5% (range 0.09 to 7%) of lymphocytes and were involving mostly CD4⁺T-lymphocytes representing a median of 73% [range 59 to 89] of IFN-γ⁺ CD3T-lymphocytes . Accordingly the proportion of IFN-γ⁺ cells was enriched in peripheral CD4T-lymphocytes with a median of 1.6% (range 0.8 to 15.5%) (FIG. 7) of IFN-γ⁺ tuberculin-specific CD4⁺T-lymphocytes. In addition, all co-produced TNF-α but not IL-2. Finally those tuberculin-specific CD4⁺T-lymphocytes displayed an extremely homogeneous differentiation profile of activated (99% [38-99] HLADR⁺) effector memory (84% [79-89] CD45RA⁻CD27⁻) IFN-γ⁺TNF-α⁺IL-2⁻T-lymphocytes.

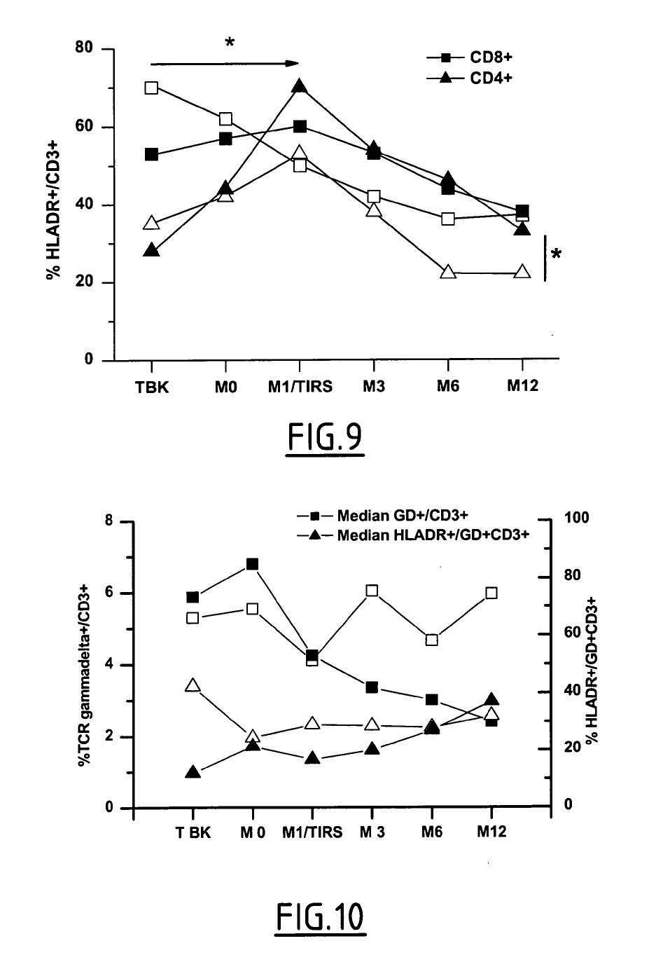

This massive involvement of activated tuberculin-specific CD4T-lymphocytes was associated with changes in bystander CD4⁺ and CD8⁺HLA⁻DR⁺ activated T-lymphocytes proportions (FIG. 9). An increase in proportions of activated CD4⁺ and CD8⁺T-lymphocytes was observed in all patients about a month after HAART onset as described during immune restoration. However the magnitude of those cells significantly differed at peak between IRS and non-IRS patients 34% [24;42] vs 18% [7;25] (p=0.02) and 10% [2;20] vs. 0 [0;7], (p=0.01) of CD4⁺ and CD8⁺T-lymphocytes respectively. CD4⁺HLADR⁺T-lymphocytes remained significantly higher during the follow-up (p=0.02) (FIG. 9). In contrast, CD25 expression on CD4T-lymphocytes remained lower in IRS patients (p=0.023, general linear model) although there was no difference in CD4⁺CD25⁺HLADR⁺ activated cells or as CD25^{hi}T-lymphocytes at time of IRS nor afterwards.

Finally the Vδ2⁺ γδ⁺T-lymphocytes increased in IRS patients after HAART onset compared to non-IRS (p=0.02) (FIGS. 10 and 11). This increase occurred either after or between time of IRS and M₃ for 6 patients or between M₆ and M₁₂ for the 5 others. The inhibitory receptors (CD158⁺ or CD94⁺) expression remained significantly lower in IRS patients after HAART on those Vδ2⁺ γδ⁺T-lymphocytes for CD158 and γδ⁺T-lymphocytes for CD94 (FIG. 3 and FIG. 4) with a median at peak of 6% [4;10] CD158⁺ Vδ2⁺ and 31% [26;43] CD94⁺ γδ⁺T-lymphocytes in those IRS patients. No change was detected in the activation status of those cells as measured by HLA-DR. Finally, in one patient it could be shown that the Vδ2⁺ cells at time of IRS involved both RA-27⁺ and RA-27⁻ cells.

In conclusion IRS is associated with expansion of activated effector memory CD4T-lymphocytes specific for mycobacteria and of Vδ2⁺T-lymphocytes.

This study shows that IRS patients differ from non-IRS patients at baseline by a lower expression of inhibitory receptors on γδ⁺T-lymphocytes and at time of IRS by an increase in cell populations involved in defence against mycobacteria, i.e., Vδ2⁺ and CD4 Th1 effector memory T-lymphocytes.

In all tested patients the tuberculin-specific T-lymphocytes were massively or uniquely involving CD4T-lymphocytes both at baseline and during IRS. Those cells had all characteristics of activated effector memory T-lymphocytes both phenotypically (CD45RA⁻CD27⁻) and functionally. The mechanism of this expansion may be redistribution or proliferation as already shown in immune restoration after HAART (Li et al. (1998) Lancet 351:1682-1686, Autran et al. (1997) Science 277:112-116).

A defect was observed in HIV-TB co-infection of the LTγδ Vδ2⁺ sub-population associated with the expansion of the usual tissular Vδ2⁻ sub-population of γδT-lymphocytes in peripheral blood. The quantitative and qualitative recovery of effector Vδ2⁺T-lymphocytes at time of IRS could be shown here, comforting the implication of these cells in the inflammatory syndrome. In addition, the significantly lower level of KIR⁺ and NKR lectin-like CD94⁺ γδT-lymphocytes in IRS versus non-IRS also argues in favour of differences in the modulation of their signalling pathways. Indeed, this functional analysis suggested that these receptors should mediate negative signals in accordance to the inhibitory function of NKGD2A in Vδ2⁺ during TB (Boullier et al. (1998) Eur J Immunol 28:3399-3410). As almost 90% of these cells are specific of the mycobacterial-antigen IPP, the lack of TCR-activation inhibitors may explain the amplification of the inflammatory syndrome and its deregulation.

Finally, the only parameter that distinguishes at baseline in this study, patients that will develop IRS from those that will not, involve the KIR.

Thus, IRS is associated with an increase in activated effector-memory CD4T-lymphocytes specific for tuberculin and in Vδ2⁺ KIR⁻ γδ⁺T-lymphocytes. This latter characteristic is already present at baseline and allows to distinguish patients who will develop IRS from those who will not.

1. A method of prognosing and/or diagnosing a M. tuberculosis-Immune restoration syndrome (TB-IRS) in an individual infected with M. tuberculosis, and optionally with HIV, comprising the steps consisting of:
a) determining the percentage or the concentration of γδ⁺ or γδ⁺Vδ2⁺T-lymphocytes which express a inhibitory KIR receptor in a biological sample of said individual;
b) comparing said percentage or concentration of γδ⁺ or γδ⁺Vδ2⁺T-lymphocytes expressing an inhibitory KIR receptor to a predetermined value; and
c) characterizing the individual's risk of developing a TB-IRS or determining if the individual suffers from TB-IRS based upon the comparison of said percentage or concentration of γδ⁺ or γδ⁺Vδ2⁺T-lymphocytes expressing an inhibitory KIR receptor with the predetermined value.

2. The method according to claim 1, wherein, in step a), a percentage or a concentration of γδ⁺ or γδ⁺Vδ2⁺T-lymphocytes which express one type of inhibitory KIR receptor is determined.

3. The method according to claim 1, wherein, in step a), a percentage or a concentration of γδ⁺ or γδ⁺Vδ2⁺T-lymphocytes which express a combination of two or more types of inhibitory KIR receptors is determined.

4. The method according to claim 1, wherein said inhibitory KIR receptor is selected from the group consisting of the members of the CD158 family, the members of the CD94/NKG2C family and NKG2D receptor.

5. The method according to claim 1, wherein said inhibitory KIR receptor is selected from the group consisting of CD94, CD158a, CD158b1 and CD158b2.

6. The method according to claim 1, wherein the predetermined value is a single value and a percentage or a concentration of γδ⁺ or γδ⁺Vδ2⁺T-lymphocytes expressing said inhibitory KIR receptor lower than the predetermined value is indicative of a risk of developing TB-IRS or is indicative of TB-IRS.

7. The method according to claim 1, wherein the predetermined value is a percentage or a concentration of γδ⁺ or γδ⁺Vδ2⁺T-lymphocytes expressing said inhibitory KIR receptor in a reference group of individuals infected with M. tuberculosis, and optionally with HIV who did not develop TB-IRS upon treatment with anti-TB treatment and/or Highly Active Antiretroviral Therapy (HAART).

8. The method according to claim 1, wherein the predetermined value comprises multiple ranges of percentage or concentration of γδ⁺ or γδ⁺Vδ2⁺T-lymphocytes expressing said inhibitory KIR receptor measured in a population of individuals infected with M. tuberculosis, and optionally with HIV, said population being divided into quantiles according to the percentage or concentration of γδ⁺ or γδ⁺Vδ2⁺T-lymphocytes expressing said inhibitory KIR receptor, the lowest quantiles being individuals with the highest risk of developing TB-IRS or having TB-IRS.

9. The method according to claim 1, which is performed in a biological sample of said individual infected with M. tuberculosis, and optionally with HIV, before onset of anti-TB treatment and/or HAART.

10. The method according to claim 1, which is performed in a biological sample of said individual infected with M. tuberculosis, and optionally with HIV in the course of anti-TB treatment and/or HAART.

11. The method according to claim 1, wherein said individual infected with M. tuberculosis, and optionally with HIV is treated with an anti-TB treatment.

12. The method according to claim 1, which further comprises monitoring the percentage of γδ⁺T-lymphocytes which are Vδ2⁺ in the course of anti-TB treatment and/or or concentration HAART, wherein an increased percentage or concentration of γδ⁺ or γδ⁺Vδ2⁺T-lymphocytes in the course of anti-TB treatment and/or HAART, is indicative of a risk of TB-IRS or of having TB-IRS.

13. The method according to claim 1, wherein the biological sample is selected from the group consisting of broncho-alveolar lavage (BAL) liquid, cerebrospinal fluid (CSF), ascitic fluid, pleural fluid, tissue mononuclear cells, whole blood, a blood fraction, and Peripheral Blood Mononuclear Cells (PBMCs).

14. A kit for prognosing and/or diagnosing M. tuberculosis-Immune restoration syndrome (TB-IRS) in a individual infected with M. tuberculosis, and optionally with HIV and M. tuberculosis, which comprises an antibody directed against TCRδ2 and at least one antibody directed against an inhibitory KIR receptor.

15. The kit according to claim 14, wherein said at least one antibody directed against an inhibitory KIR receptor is selected from the group consisting of an antibody directed against CD94, an antibody directed against CD158a, an antibody directed against CD158b1, an antibody directed against CD158b2, and an antibody directed against NKG2D receptor.
